(19) United States
(12) Patent Application Publication
(10) Pub. No.: US 2023/0355325 A1
(43) Pub. Date: Nov. 9, 2023

(54) REPLACEABLE ARM GUIDE AND END EFFECTOR FOR SURGICAL SYSTEMS

(71) Applicant: Mazor Robotics Ltd., Caesarea (IL)

(72) Inventors: Ziv Seemann, Beit Ytzhack (IL); Gal Barazani, Haifa (IL); Nir Ofer, Tel Aviv-Yafo (IL); Amir Keret, Atlit (IL); Dvir Kadshai, Tel Aviv (IL); Adam David Glaser, Collierville, TN (US); Daniel Paxton Wall, Cordova, TN (US)

(21) Appl. No.: 17/735,901

(22) Filed: May 3, 2022

(51) Int. Cl. A61B 34/30; A61B 46/10; A61B 90/50

(52) U.S. Cl. CPC A61B 34/30; A61B 46/10; A61B 90/50; A61B 2034/306

(57) ABSTRACT

A surgical system according to at least one embodiment of the present disclosure includes an interface block disposed at a distal end of a robot arm and an end-effector block that attaches to the interface block via a nut. For example, the interface block may include a threaded rod that extends a distance from a mount surface of the interface block. Accordingly, the end-effector block may include a mount hole that passes through the end-effector block, where the nut threadedly engages with the threaded rod through the mount hole to clamp the end-effector block against the interface block. In some examples, a sterile drape may be used to provide a sterile barrier between the surgical system and a patient. Additionally, a liner plate may be used with the surgical system, such that an area of the sterile drape is disposed between the liner plate and the mount surface.

### BACKGROUND

The present disclosure is generally directed to surgical systems, and relates more particularly to robotic surgical devices.

Surgical robots may assist a surgeon or other medical provider in carrying out a surgical procedure or may complete one or more surgical procedures autonomously. Providing controllable linked articulating members allows a surgical robot to reach areas of a patient anatomy during various medical procedures.

### BRIEF SUMMARY

Example aspects of the present disclosure include:

A surgical system, comprising: a robot arm comprising a proximal end and a distal end; an interface block disposed at the distal end, the interface block comprising: a mount surface; a threaded rod extending a distance from the mount surface; and a set of kinematic attachment features disposed on the mount surface around the threaded rod; and an end-effector block, comprising: a set of kinematic connectors that are arranged to engage with the set of kinematic attachment features of the interface block; and a mount hole passing through the end-effector block, wherein the end-effector block is moveable between an attached state and a detached state, wherein, in the attached state, the set of kinematic connectors are engaged with the set of kinematic attachment features, the threaded rod is disposed in the mount hole, and a nut threadedly engages with the threaded rod clamping the end-effector block against the interface block.

Any of the aspects herein, wherein, in the detached state, the nut is removed and the end-effector block is separated from the interface block such that the threaded rod remains attached to the interface block and is no longer disposed in the mount hole.

Any of the aspects herein, further comprising: a sterile drape that covers the robot arm, wherein the threaded rod pierces a controlled portion of the sterile drape; and a liner plate comprising a clearance hole that is arranged such that the threaded rod passes through the clearance hole and an area of the sterile drape is disposed between the liner plate and the mount surface, and wherein the end-effector block is moveable between the attached state and the detached state without removing the threaded rod from the interface block and without exposing an environment inside the sterile drape to an environment outside of the sterile drape.

Any of the aspects herein, wherein the controlled portion of the sterile drape is disposed within a periphery of the interface block.

Any of the aspects herein, wherein the sterile drape further comprises: a gasket attached to the sterile drape, the gasket surrounding the controlled portion of the sterile drape and an outer circumference of the threaded rod.

Any of the aspects herein, wherein the gasket is formed in a flat circular ring shape, and wherein at least one flat surface of the gasket comprises an adhesive layer.

Any of the aspects herein, wherein the gasket is attached to the sterile drape on a side of the sterile drape facing the mount surface, and wherein the adhesive layer is disposed in contact with the mount surface sealing the environment inside the sterile drape and around the gasket from the environment outside of the sterile drape.

Any of the aspects herein, wherein the liner plate is made from a flat metal plate material corresponding to at least one of aluminum, copper, titanium, cobalt-chrome, and stainless steel.

Any of the aspects herein, wherein the liner plate is made from a flat polymer plate material.

Any of the aspects herein, wherein the mount hole comprises an unthreaded clearance hole and counterbore.

A robot end-effector mount system, comprising: an interface block, comprising: a mount surface; a threaded rod protruding from the mount surface; and an end-effector block, comprising: a body; a tool receiving aperture; and a mount hole passing through the body, wherein the end-effector block is moveable between an attached state and a detached state with the interface block, wherein, in the attached state, the threaded rod is disposed in the mount hole and a nut threadedly engages with the threaded rod clamping the end-effector block against the interface block, and wherein the end-effector block is moveable between the attached state and the detached state without removing the threaded rod from the interface block.

Any of the aspects herein, wherein, in the detached state, the nut is removed and the end-effector block is separated from the interface block such that the threaded rod remains attached to the interface block and is no longer disposed in the mount hole.

Any of the aspects herein, further comprising: a sterile drape that covers the interface block, wherein the sterile drape is pierced at a controlled portion by the threaded rod; and a liner plate comprising a clearance hole that is arranged such that the threaded rod passes through the clearance hole and an area of the sterile drape is disposed between the liner plate and the mount surface, and wherein the end-effector block is moveable between the attached state and the detached state without exposing an environment inside the sterile drape on a side of the sterile drape facing the mount surface to an environment outside of the sterile drape on a side of the sterile drape facing the end-effector block.

Any of the aspects herein, wherein the controlled portion of the sterile drape is disposed within an outer edge of the interface block.

Any of the aspects herein, wherein the sterile drape further comprises: a gasket attached to the sterile drape, wherein the gasket surrounds the controlled portion of the sterile drape and an outer circumference of the threaded rod, wherein the gasket is formed in a flat circular ring shape, and wherein at least one flat surface of the gasket comprises an adhesive layer.

Any of the aspects herein, wherein the gasket is formed in a flat circular ring shape, and wherein at least one flat surface of the gasket comprises an adhesive layer.

Any of the aspects herein, wherein the gasket is attached to the sterile drape on a side of the sterile drape facing the mount surface, and wherein the adhesive layer is disposed in contact with the mount surface sealing the environment inside the sterile drape and around the gasket from the environment outside of the sterile drape.

Any of the aspects herein, wherein the liner plate is made from a flat metal plate material corresponding to at least one of aluminum, copper, titanium, cobalt-chrome, and stainless steel.

Any of the aspects herein, wherein the liner plate is made from a flat polymer plate material.

A robot end-effector mount system, comprising: an end-effector block, comprising: a body; a tool receiving aperture; and a mount hole passing through the body, wherein the end-effector block is moveable between an attached state and a detached state with an interface block, wherein, in the attached state, a threaded rod is disposed in the mount hole and a nut threadedly engages with the threaded rod clamping the end-effector block against the interface block, and wherein the end-effector block is moveable between the attached state and the detached state without removing the threaded rod from the interface block.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a block diagram of a system according to at least one embodiment of the present disclosure;

FIG. 2A is a perspective diagram of a robotic surgical system according to at least one embodiment of the present disclosure;

FIG. 2B is a perspective diagram of a robotic surgical system according to at least one embodiment of the present disclosure;

FIG. 3 is a diagram of an interface block according to at least one embodiment of the present disclosure;

FIG. 4 is a diagram of the interface block with a liner plate attached according to at least one embodiment of the present disclosure; and

FIG. 5A is a perspective view of a surgical system according to at least one embodiment of the present disclosure;

FIG. 5B is a front view of the surgical system according to at least one embodiment of the present disclosure;

FIG. 6 is an assembly diagram for attaching the end-effector block to the interface block according to at least one embodiment of the present disclosure; and

FIG. 7 is a flowchart of a method for selectively coupling the end-effector block to a robot arm according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia GeForce RTX 2000-series processors, Nvidia GeForce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

During a robotic surgical procedure, a robotic surgical system needs to have different components that are sterile. For example, the robotic surgical system may include end-effectors, guiding tools, etc. that need to be kept sterile. Existing end-effectors may be manufactured specifically to different screw system diameters (e.g., 4.5 millimeters (mm), 6.5 mm, 10.5 mm, etc.). That is, the end-effectors may be connected to robotic surgical systems via screws, where the screws have a specific diameter according to a screw system diameter used for the robotic surgical procedure. However, the screw system cannot be replaced during a robotic surgical procedure (e.g., a counterbore for accepting the screws stays constant), so all end-effectors needed to perform the robotic surgical procedure have to connect to the robotic surgical system using same diameter-sized screws. Additionally or alternatively, the screw system diameter of a robotic surgical procedure may correspond to a same diameter of screws inserted into body parts of a patient, where same size screws are used for the robotic surgical procedure. For example, the end-effectors may include or use guiding tools for drilling having the different diameters. The end-effectors may also have additional capabilities (e.g., drilling, cutting, etc.) that need to take the screw system diameter into account. Accordingly, if the robotic surgical procedure has multiple levels, a surgeon may need to compromise on a screw system to fit all vertebras (e.g., different sized screws could be used for different vertebrae, but a same screw system diameter may be used for all vertebras because the screw system cannot be replaced during the robotic surgical procedure).

As described herein, an end-effector block is described and provided that can be connected or disconnected from a surgical system using a nut that threads onto a threaded rod instead of using a screw. Using a nut to attach the end-effector block to the surgical system will allow for replacing of different components of the surgical system (e.g., arm guides, other cutting tools, etc.) without risking a compromise of a sterile barrier between the surgical system, a patient, and the surrounding environment. Additionally, using a nut instead of a screw to attach end-effector blocks may support design of tools that can include more features other than currently offered.

In some examples, the end-effector block may connect to a liner plate on a contact area (e.g., mount surface) of an interface block disposed at a distal end of a robot arm (e.g., with kinematic coupling) that will allow forces to be distributed more evenly across the contact area and hold the forces without tearing components of the surgical system (e.g., sterile drapes). The liner plate may be a thin metal plate (e.g., made of aluminum, copper, stainless steel, titanium, cobalt-chrome, etc., or a different metal not explicitly disclosed herein). Additionally or alternatively, the liner plate may be a polymer or plastic material (e.g., with a large elongation of the plastic material).

Embodiments of the present disclosure provide technical solutions to one or more of the problems of (1) using screws to attach end-effector blocks in a surgical system, (2) compromising sterile barriers, and (3) limiting which tools can be used in or connected to a surgical system. For example, the surgical system and method of interconnecting the end-effector block described herein allows an arm guide/end-effector to be connected/disconnected using a nut-to-rod interface rather than an external screw-in-hole interface. This reversed interface allows changeover of an arm guide without compromising a sterile barrier between the patient and the robot. In addition, the liner plate provides a rigid interface that prevents wear of the sterile drape at the interconnection point and allows forces to be distributed evenly across a mount surface of the robot arm. Among other things, the idea allows an arm guide or end effector to be replaced during a procedure maintaining sterility (e.g., allowing for use of several screw systems to be used in a same spinal surgery) and reducing the number of tools (e.g., less types of cannulas) used in an operating room.

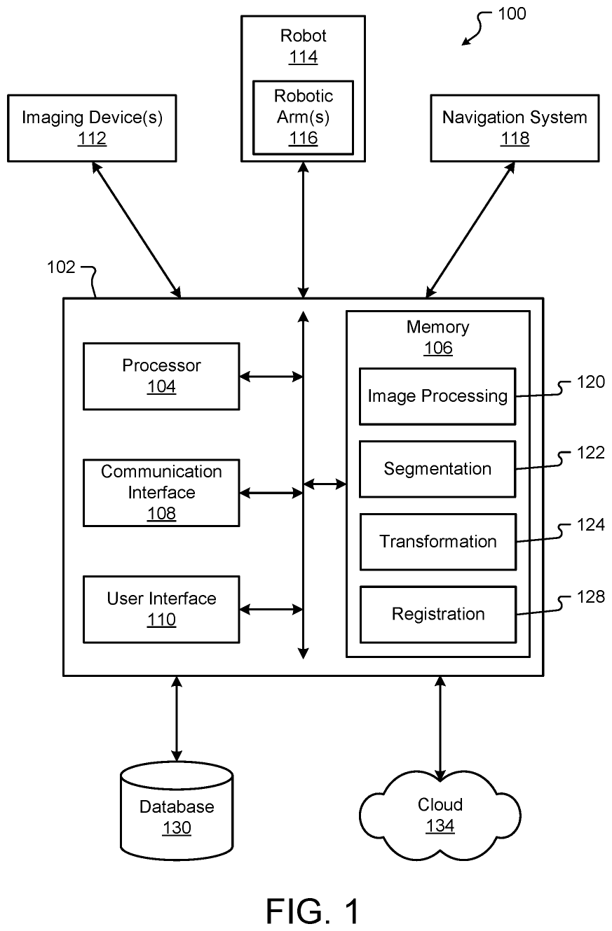

Turning first to FIG. 1, a block diagram of a system 100 according to at least one embodiment of the present disclosure is shown. The system 100 may be used to selectively couple an end-effector block to a robot arm in a sterile environment. In some examples, the system 100 may control, pose, and/or otherwise manipulate a surgical mount system, a surgical arm, and/or surgical tools attached thereto and/or carry out one or more other aspects of one or more of the methods disclosed herein. The system 100 comprises a computing device 102, one or more imaging devices 112, a robot 114, a navigation system 118, a database 130, and/or a cloud or other network 134. Systems according to other embodiments of the present disclosure may comprise more or fewer components than the system 100. For example, the system 100 may not include the imaging device 112, the robot 114, the navigation system 118, one or more components of the computing device 102, the database 130, and/or the cloud 134.

The computing device 102 comprises a processor 104, a memory 106, a communication interface 108, and a user interface 110. Computing devices according to other embodiments of the present disclosure may comprise more or fewer components than the computing device 102.

The processor 104 of the computing device 102 may be any processor described herein or any similar processor. The processor 104 may be configured to execute instructions stored in the memory 106, which instructions may cause the processor 104 to carry out one or more computing steps utilizing or based on data received from the imaging device 112, the robot 114, the navigation system 118, the database 130, and/or the cloud 134.

The memory 106 may be or comprise RAM, DRAM, SDRAM, other solid-state memory, any memory described herein, or any other tangible, non-transitory memory for storing computer-readable data and/or instructions. The memory 106 may store information or data useful for completing, for example, any step of the method 700 described herein, or of any other methods. The memory 106 may store, for example, instructions and/or machine learning models that support one or more functions of the robot 114. For instance, the memory 106 may store content (e.g., instructions and/or machine learning models) that, when executed by the processor 104, enable image processing 120, segmentation 122, transformation 124, and/or registration 128. Such content, if provided as in instruction, may, in some embodiments, be organized into one or more applications, modules, packages, layers, or engines. Alternatively or additionally, the memory 106 may store other types of content or data (e.g., machine learning models, artificial neural networks, deep neural networks, etc.) that can be processed by the processor 104 to carry out the various method and features described herein. Thus, although various contents of memory 106 may be described as instructions, it should be appreciated that functionality described herein can be achieved through use of instructions, algorithms, and/or machine learning models. The data, algorithms, and/or instructions may cause the processor 104 to manipulate data stored in the memory 106 and/or received from or via the imaging device 112, the robot 114, the database 130, and/or the cloud 134.

The computing device 102 may also comprise a communication interface 108. The communication interface 108 may be used for receiving image data or other information from an external source (such as the imaging device 112, the robot 114, the navigation system 118, the database 130, the cloud 134, and/or any other system or component not part of the system 100), and/or for transmitting instructions, images, or other information to an external system or device (e.g., another computing device 102, the imaging device 112, the robot 114, the navigation system 118, the database 130, the cloud 134, and/or any other system or component not part of the system 100). The communication interface 108 may comprise one or more wired interfaces (e.g., a USB port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some embodiments, the communication interface 108 may be useful for enabling the device 102 to communicate with one or more other processors 104 or computing devices 102, whether to reduce the time needed to accomplish a computing-intensive task or for any other reason.

The computing device 102 may also comprise one or more user interfaces 110. The user interface 110 may be or comprise a keyboard, mouse, trackball, monitor, television, screen, touchscreen, and/or any other device for receiving information from a user and/or for providing information to a user. The user interface 110 may be used, for example, to receive a user selection or other user input regarding any step of any method described herein. Notwithstanding the foregoing, any required input for any step of any method described herein may be generated automatically by the system 100 (e.g., by the processor 104 or another component of the system 100) or received by the system 100 from a source external to the system 100. In some embodiments, the user interface 110 may be useful to allow a surgeon or other user to modify instructions to be executed by the processor 104 according to one or more embodiments of the present disclosure, and/or to modify or adjust a setting of other information displayed on the user interface 110 or corresponding thereto.

Although the user interface 110 is shown as part of the computing device 102, in some embodiments, the computing device 102 may utilize a user interface 110 that is housed separately from one or more remaining components of the computing device 102. In some embodiments, the user interface 110 may be located proximate one or more other components of the computing device 102, while in other embodiments, the user interface 110 may be located remotely from one or more other components of the computer device 102.

The imaging device 112 may be operable to image anatomical feature(s) (e.g., a bone, veins, tissue, etc.) and/or other aspects of patient anatomy to yield image data (e.g., image data depicting or corresponding to a bone, veins, tissue, etc.). “Image data” as used herein refers to the data generated or captured by an imaging device 112, including in a machine-readable form, a graphical/visual form, and in any other form. In various examples, the image data may comprise data corresponding to an anatomical feature of a patient, or to a portion thereof. The image data may be or comprise a preoperative image, an intraoperative image, a postoperative image, or an image taken independently of any surgical procedure. In some embodiments, a first imaging device 112 may be used to obtain first image data (e.g., a first image) at a first time, and a second imaging device 112 may be used to obtain second image data (e.g., a second image) at a second time after the first time. The imaging device 112 may be capable of taking a 2D image or a 3D image to yield the image data. The imaging device 112 may be or comprise, for example, an ultrasound scanner (which may comprise, for example, a physically separate transducer and receiver, or a single ultrasound transceiver), an O-arm, a C-arm, a G-arm, or any other device utilizing X-ray-based imaging (e.g., a fluoroscope, a CT scanner, or other X-ray machine), a magnetic resonance imaging (MM) scanner, an optical coherence tomography (OCT) scanner, an endoscope, a microscope, an optical camera, a thermographic camera (e.g., an infrared camera), a radar system (which may comprise, for example, a transmitter, a receiver, a processor, and one or more antennae), or any other imaging device 112 suitable for obtaining images of an anatomical feature of a patient. The imaging device 112 may be contained entirely within a single housing, or may comprise a transmitter/emitter and a receiver/detector that are in separate housings or are otherwise physically separated.

In some embodiments, the imaging device 112 may comprise more than one imaging device 112. For example, a first imaging device may provide first image data and/or a first image, and a second imaging device may provide second image data and/or a second image. In still other embodiments, the same imaging device may be used to provide both the first image data and the second image data, and/or any other image data described herein. The imaging device 112 may be operable to generate a stream of image data. For example, the imaging device 112 may be configured to operate with an open shutter, or with a shutter that continuously alternates between open and shut so as to capture successive images. For purposes of the present disclosure, unless specified otherwise, image data may be considered to be continuous and/or provided as an image data stream if the image data represents two or more frames per second.

The robot 114 may be any surgical robot or surgical robotic system. The robot 114 may be or comprise, for example, the Mazor X™ Stealth Edition robotic guidance system. The robot 114 may be configured to position the imaging device 112 at one or more precise position(s) and orientation(s), and/or to return the imaging device 112 to the same position(s) and orientation(s) at a later point in time. The robot 114 may additionally or alternatively be configured to manipulate a surgical tool (whether based on guidance from the navigation system 118 or not) to accomplish or to assist with a surgical task. In some embodiments, the robot 114 may be configured to hold and/or manipulate an anatomical element during or in connection with a surgical procedure. The robot 114 may comprise one or more robotic arms 116. In some embodiments, the robotic arm 116 may comprise a first robotic arm and a second robotic arm, though the robot 114 may comprise more than two robotic arms. In some embodiments, one or more of the robotic arms 116 may be used to hold and/or maneuver the imaging device 112. In embodiments where the imaging device 112 comprises two or more physically separate components (e.g., a transmitter and receiver), one robotic arm 116 may hold one such component, and another robotic arm 116 may hold another such component. Each robotic arm 116 may be positionable independently of the other robotic arm. The robotic arms 116 may be controlled in a single, shared coordinate space, or in separate coordinate spaces.

The robot 114, together with the robotic arm 116, may have, for example, one, two, three, four, five, six, seven, or more degrees of freedom. Further, the robotic arm 116 may be positioned or positionable in any pose, plane, and/or focal point. The pose includes a position and an orientation. As a result, an imaging device 112, surgical tool, or other object held by the robot 114 (or, more specifically, by the robotic arm 116) may be precisely positionable in one or more needed and specific positions and orientations.

The robotic arm(s) 116 may comprise one or more sensors that enable the processor 104 (or a processor of the robot 114) to determine a precise pose in space of the robotic arm (as well as any object or element held by or secured to the robotic arm).

In some embodiments, reference markers (e.g., navigation markers) may be placed on the robot 114 (including, e.g., on the robotic arm 116), the imaging device 112, or any other object in the surgical space. The reference markers may be tracked by the navigation system 118, and the results of the tracking may be used by the robot 114 and/or by an operator of the system 100 or any component thereof. In some embodiments, the navigation system 118 can be used to track other components of the system (e.g., imaging device 112) and the system can operate without the use of the robot 114 (e.g., with the surgeon manually manipulating the imaging device 112 and/or one or more surgical tools, based on information and/or instructions generated by the navigation system 118, for example).

The navigation system 118 may provide navigation for a surgeon and/or a surgical robot during an operation. The navigation system 118 may be any now-known or future-developed navigation system, including, for example, the Medtronic StealthStation™ S8 surgical navigation system or any successor thereof. The navigation system 118 may include one or more cameras or other sensor(s) for tracking one or more reference markers, navigated trackers, or other objects within the operating room or other room in which some or all of the system 100 is located. The one or more cameras may be optical cameras, infrared cameras, or other cameras. In some embodiments, the navigation system 118 may comprise one or more electromagnetic sensors. In various embodiments, the navigation system 118 may be used to track a position and orientation (e.g., a pose) of the imaging device 112, the robot 114 and/or robotic arm 116, and/or one or more surgical tools (or, more particularly, to track a pose of a navigated tracker attached, directly or indirectly, in fixed relation to the one or more of the foregoing). The navigation system 118 may include a display for displaying one or more images from an external source (e.g., the computing device 102, imaging device 112, or other source) or for displaying an image and/or video stream from the one or more cameras or other sensors of the navigation system 118. In some embodiments, the system 100 can operate without the use of the navigation system 118. The navigation system 118 may be configured to provide guidance to a surgeon or other user of the system 100 or a component thereof, to the robot 114, or to any other element of the system 100 regarding, for example, a pose of one or more anatomical elements, whether or not a tool is in the proper trajectory, and/or how to move a tool into the proper trajectory to carry out a surgical task according to a preoperative or other surgical plan.

The database 130 may store information that correlates one coordinate system to another (e.g., one or more robotic coordinate systems to a patient coordinate system and/or to a navigation coordinate system). The database 130 may additionally or alternatively store, for example, one or more surgical plans (including, for example, pose information about a target and/or image information about a patient's anatomy at and/or proximate the surgical site, for use by the robot 114, the navigation system 118, and/or a user of the computing device 102 or of the system 100); one or more images useful in connection with a surgery to be completed by or with the assistance of one or more other components of the system 100; and/or any other useful information. The database 130 may be configured to provide any such information to the computing device 102 or to any other device of the system 100 or external to the system 100, whether directly or via the cloud 134. In some embodiments, the database 130 may be or comprise part of a hospital image storage system, such as a picture archiving and communication system (PACS), a health information system (HIS), and/or another system for collecting, storing, managing, and/or transmitting electronic medical records including image data.

The cloud 134 may be or represent the Internet or any other wide area network. The computing device 102 may be connected to the cloud 134 via the communication interface 108, using a wired connection, a wireless connection, or both. In some embodiments, the computing device 102 may communicate with the database 130 and/or an external device (e.g., a computing device) via the cloud 134.

The system 100 or similar systems may be used, for example, to carry out one or more aspects of any of the method 700 described herein. The system 100 or similar systems may also be used for other purposes.

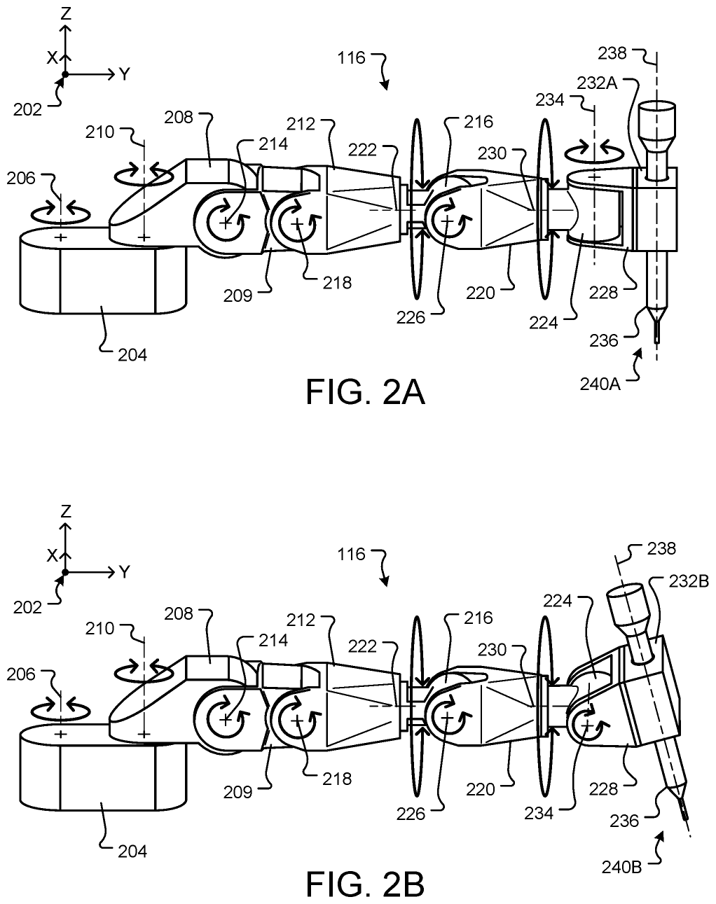

Referring now to FIGS. 2A and 2B, perspective diagrams of a robotic surgical system with different end effector 240A, 240B mount positions are shown in accordance with examples of the present disclosure. More specifically, FIGS. 2A and 2B show the robotic arm 116 of the robot 114 connected to an end effector 240A, 240B holding a surgical tool 236. While shown as a single surgical tool 236 in FIGS. 2A and 2B, the surgical tool 236 may correspond to different surgical tools used between operations in a surgical application. For instance, a first surgical tool 236 may include a direction-specific blade that may require a specific rotational alignment and placement in the tool block 232A, 232B, while another surgical tool 236 may include a unidirectional cutting tool that is independent of rotational alignment in the tool block 232A, 232B.

Features of the robot 114 and/or robotic arm 116 may be described in conjunction with a coordinate system 202. The coordinate system 202, as shown in FIGS. 2A and 2B, includes three-dimensions comprising an X-axis, a Y-axis, and a Z-axis. Additionally or alternatively, the coordinate system 202 may be used to define planes (e.g., the XY-plane, the XZ-plane, and the YZ-plane) of the robot 114 and/or robotic arm 116. These planes may be disposed orthogonal, or at 90 degrees, to one another. While the origin of the coordinate system 202 may be placed at any point on or near the components of the robot 114, for the purposes of description, the axes of the coordinate system 202 are always disposed along the same directions from figure to figure, whether the coordinate system 202 is shown or not. In some examples, reference may be made to dimensions, angles, directions, relative positions, and/or movements associated with one or more components of the robot 114 and/or robotic arm 116 with respect to the coordinate system 202. For example, the width of the robotic arm 116 (e.g., running from the side shown in the foreground to the side in the background, into the page) may be defined as a dimension along the X-axis of the coordinate system 202, the height of the robotic arm 116 may be defined as a dimension along the Z-axis of the coordinate system 202, and the length of the robotic arm 116 (e.g., running from a proximal end at the first link 204 to a distal end at the seventh link 224, etc.) may be defined as a dimension along the Y-axis of the coordinate system 202. Additionally or alternatively, the height of the system 100 may be defined as a dimension along the Z-axis of the coordinate system 202, a reach of the robotic arm 116 may be defined as a dimension along the Y-axis of the coordinate system 202, and a working area of the robotic arm 116 may be defined in the XY-plane with reference to the corresponding axes of the coordinate system 202.

The robotic arm 116 may be comprised of a number of links 204, 208, 209, 212, 216, 220, 224 that interconnect with one another at respective axes of rotation 206, 210, 214, 218, 222, 226, 230, 234, or joints. There may be more or fewer links 204, 208, 209, 212, 216, 220, 224 and/or axes of rotation 206, 210, 214, 218, 222, 226, 230, 234 than are shown in FIGS. 2A and 2B. In any event, the robotic arm 116 may have a first link 204 disposed at a proximal end of the robotic arm 116 and an end mount flange 228 disposed furthest from the proximal end at a distal end of the robotic arm 116. The first link 204 may correspond to a base of the robotic arm 116. In some examples, the first link 204 may rotate about first rotation axis 206. A second link 208 may be connected to the first link 204 at a second rotation axis 210, or joint. The second link 208 may rotate about the second rotation axis 210. In one example, the first rotation axis 206 and the second rotation axis 210 may be arranged parallel to one another. For instance, the first rotation axis 206 and the second rotation axis 210 are shown extending along the Z-axis in a direction perpendicular to the XY-plane.

The robotic arm 116 may comprise a third link 209 that is rotationally interconnected to the second link 208 via the third rotation axis 214, or joint. The third rotation axis 214 is shown extending along the X-axis, or perpendicular to the first rotation axis 206 and second rotation axis 210. In this position, when the third link 209 is caused to move (e.g., rotate relative to the second link 208), the third link 209 (and the components of the robotic arm 116 extending from the third link 209) may be caused to move into or out of the XY-plane. The fourth link 212 is shown rotationally interconnected to the third link 209 via the fourth rotation axis 218, or joint. The fourth rotation axis 218 is arranged parallel to the third rotation axis 214. The fourth rotation axis 218 extends along the X-axis allowing rotation of the fourth link 212 into and out of the XY-plane.

In some examples, the robotic arm 116 may comprise one or more wrists 216, 224. The fifth link 216, or wrist, is shown rotationally interconnected to the fourth link 212 via a fifth rotation axis 222, or wrist joint. The fifth rotation axis 222 is shown extending along the Y-axis, which is perpendicular to the X-axis and the Z-axis. During operation of the robot 114, causing the fifth link 216 to rotate about the fifth rotation axis 222 may cause the components of the robotic arm 116 distal the joint at the fifth rotation axis 222 (e.g., the fifth link 216, the sixth link 220, the seventh link 224, the end mount flange 228, and the end effector 240A, 240B, etc.) to rotate about the Y-axis.

The sixth link 220 is rotationally interconnected to the fifth link 216 via the sixth rotation axis 226. The sixth rotation axis 226 extends along the X-axis and provides for rotation of the sixth link 220 relative to the fifth link 216 (e.g., into and out of the XY-plane in the position shown).

The seventh link 224, or wrist, is shown rotationally interconnected to the sixth link 220 via a seventh rotation axis 230, or wrist joint. The seventh rotation axis 230 is shown extending along the Y-axis (e.g., perpendicular to the X-axis and the Z-axis). During operation of the robot 114, causing the seventh link 224 to rotate about the seventh rotation axis 230 may cause the components of the robotic arm 116 distal the joint at the seventh rotation axis 230 (e.g., the end mount flange 228, and the end effector 240A, 240B, etc.) to rotate about the Y-axis.

Located at the distal end of the robotic arm 116, an end mount flange 228 may be rotationally interconnected to the end mount flange 228 via an eighth, or mount flange rotation, axis 234. In FIG. 2A, the seventh link 224 is positioned rotationally about the seventh rotation axis 230 such that the end mount flange 228 is oriented where the mount flange rotation axis 234 is extending along the Z-axis. In FIG. 2B, the seventh link 224 is positioned rotationally about the seventh rotation axis 230 such that the end mount flange 228 is oriented where the mount flange rotation axis 234 is extending along the X-axis. In some examples, at least the seventh link 224 may be rotated about the seventh rotation axis 230 to move between the end mount flange 228 position shown in FIG. 2A and the end mount flange 228 position shown in FIG. 2B, or vice versa. The end mount flange 228 and the mount flange rotation axis 234 may be the last movable (e.g., motor actuated, etc.) link and joint of the robotic arm 116. Moving between these two positions of the end mount flange 228 allows a particular end effector 240A, 240B to be attached and manipulated, or operated, according to a corresponding movement profile (e.g., range and limits) or set of kinematic solutions for the robot 114 (e.g., the robotic arm 116 and the surgical tool 236, etc.).

FIG. 2A shows first movement kinematics for the robotic arm 116 when the first tool block 232A of the first end effector 240A disposes the surgical tool axis 238 parallel to the mount flange rotation axis 234. In the position shown in FIG. 2A, rotation into and/or out of the XY-plane between the seventh link 224 and the first end effector 240A is prevented. This position and arrangement may be ideal for applications (e.g., operations, procedures, etc.) where an end rotational position of the surgical tool 236 may need to be maintained for the robotic arm 116. For example, the surgical tool 236 in the first end effector 240A may correspond to an imaging device that may need to be maintained in a particular nonrotational position relative to a patient during imaging (e.g., where an imaging plane of the surgical tool 236 should be maintained parallel to the XY-plane as other joints of the robotic arm 116 move the distal end closer to or further from the proximal end). In this case, the corresponding arrangement of the surgical tool axis 238 (e.g., parallel to the mount flange rotation axis 234) associated with the first end effector 240A may be preferred. In another example, rotation of the surgical tool 236 into, or out of, the XY-plane may need to be prevented to ensure accuracy of movement along the Y-axis, in the XY-plane, and/or the like. Additionally or alternatively, a distance between a reference plane and an end of the surgical tool 236 (e.g., along the Z-axis) may need to remain constant during operation of the robot 114. In any of these cases, the position and arrangement shown in conjunction with FIG. 2A (e.g., preventing end rotation relative to the XY-plane) may be preferred.

FIG. 2B shows second movement kinematics for the robotic arm 116 when the second tool block 232B of the second end effector 240B disposes the surgical tool axis 238 perpendicular (e.g., at 90 degrees) to the mount flange rotation axis 234. In this alternative position, the end mount flange 228 and second end effector 240B may be allowed to rotate relative to the seventh link 224. Stated another way, in this alternative position, the end mount flange 228 and second end effector 240B may be allowed to rotate into and/or out of the XY-plane (e.g., relative to seventh link 224 at the mount flange rotation axis 234). This position and arrangement may be ideal when a precise rotational movement of the surgical tool 236 at the distal end of the robotic arm 116 is desired. In contrast to the position and arrangement shown in FIG. 2A, where the closest rotation of the first end effector 240A about the X-axis is provided at the sixth rotation axis 226, the position and arrangement of FIG. 2B allows the second end effector 240B to be rotated about the X-axis about the mount flange rotation axis 234. Among other things, this position and arrangement may be used for any application where a movement of the second end effector 240B including an end rotation into and/or out of the XY-plane is desired for the surgical tool 236. Such applications may include directional cutting operations, probing movements, displacement of tissue and organs, and/or other surgical operations.

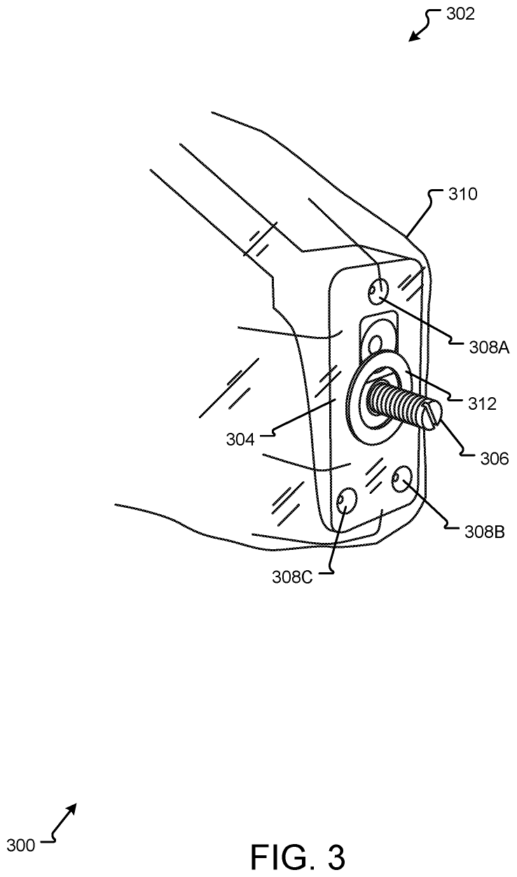

FIG. 3 shows a diagram 300 of an interface block 302 according to at least one embodiment of the present disclosure. In some examples, the interface block 302 may represent an example of an end mount flange 228 as described with reference to FIGS. 2A and 2B. For example, the interface block 302 may be disposed at a distal end of a robot arm (e.g., a robotic arm 116 as described with reference to FIG. 1).

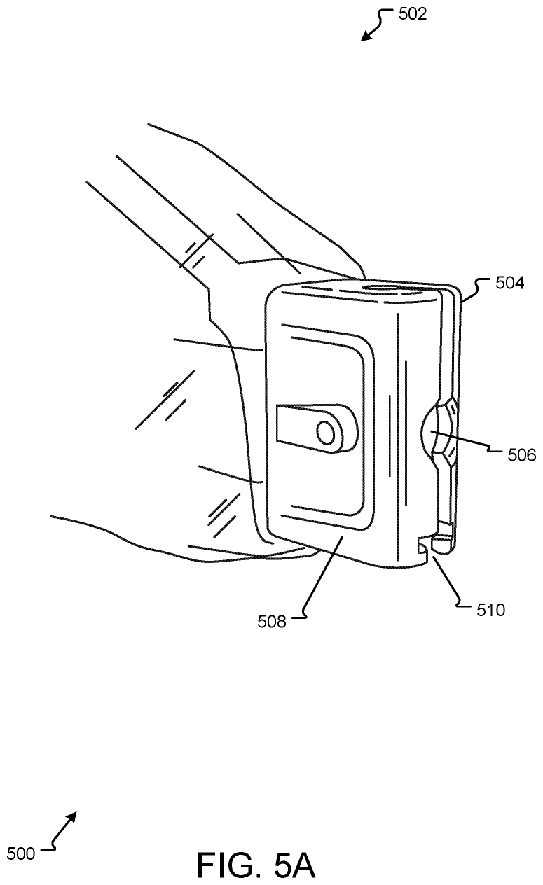

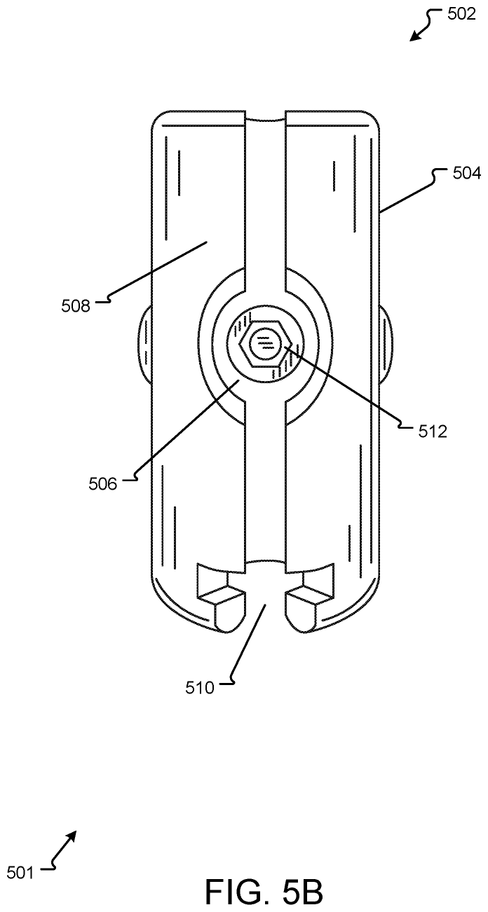

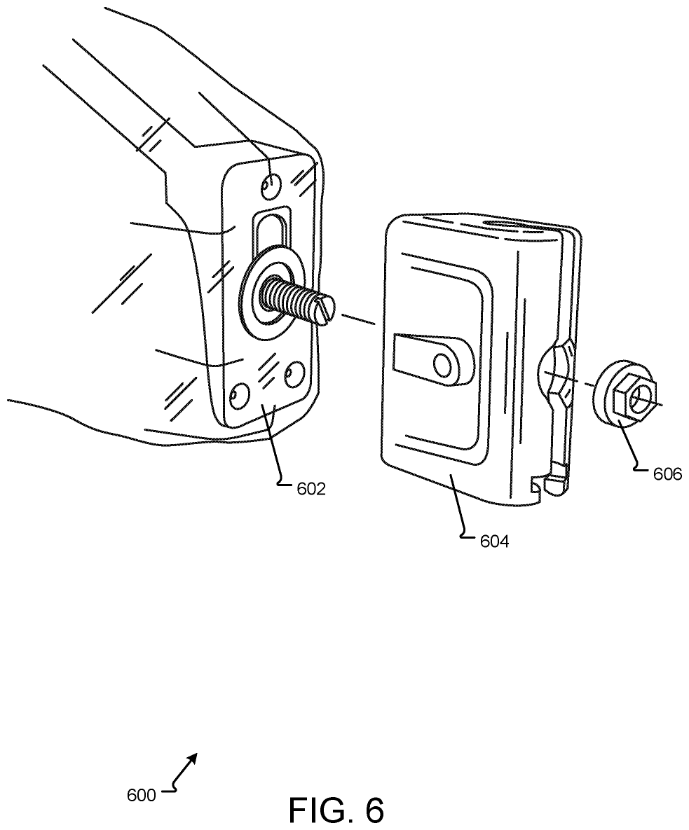

In some examples, the interface block 302 may include a mount surface 304, a threaded rod 306 extending a distance from the mount surface 304, and a set of kinematic attachment features 308 disposed on the mount surface 304 around the threaded rod 306. While three (3) kinematic attachment features 308 are shown in the example of FIG. 3 (e.g., a first kinematic attachment feature 308A, a second kinematic attachment feature 308B, and a third kinematic feature 308C), a greater or lesser number of kinematic attachment features 308 may be present on an interface block. The interface block 302 may allow for connection of an end-effector block using a nut that threads onto the threaded rod 306. The end-effector block and how the end-effector block connects to the interface block 302 using a nut with the threaded rod 306 is described in greater detail with reference to FIGS. 5A, 5B, and 6. In some examples, the set of kinematic attachment features 308 may engage with a set of kinematic connectors arranged on the end-effector block.

Additionally, a sterile drape 310 may be used as part of a surgical system of which the interface block 302 is a part. The sterile drape 310 may cover the robot arm on which the interface block 302, such that the mounting surface 304 and the set of kinematic attachment features 308 are also covered by the sterile drape 310. The sterile drape 310 may provide a sterile barrier between the patient and the robot/surgical system.

In some examples, the threaded rod 306 may pierce a controlled portion of the sterile drape 310. For example, the controlled portion of the sterile drape 310 may be disposed within a periphery of the interface block 302. Additionally, the sterile drape 310 may include a gasket 312 that is attached to the sterile drape 310, where the gasket 312 surrounds the controlled portion of the sterile drape 310 and an outer circumference of the threaded rod 306. In some examples, the gasket 312 may be formed in a flat circular ring shape, where at least one flat surface of the gasket 312 includes an adhesive layer. In some examples, the gasket 312 may be attached to the sterile drape 310 on a side of the sterile drape 310 facing the mount surface 304, where the adhesive layer is disposed in contact with the mount surface 304 sealing the environment inside the sterile drape 310 and around the gasket 312 from the environment outside the sterile drape 310. Additionally or alternatively, the gasket 312 may be attached to the sterile drape 310 on the opposite side of the sterile drape 310 (e.g., on the side outside the mount surface 304).

In some examples, the diagram 300 may represent a detached state for an end-effector block as described herein, where the nut is removed and the end-effector block is separated from the interface block 302 such that the threaded rod 306 remains attached to the interface block 302 and is not disposed in a mount hole of the end-effector block.

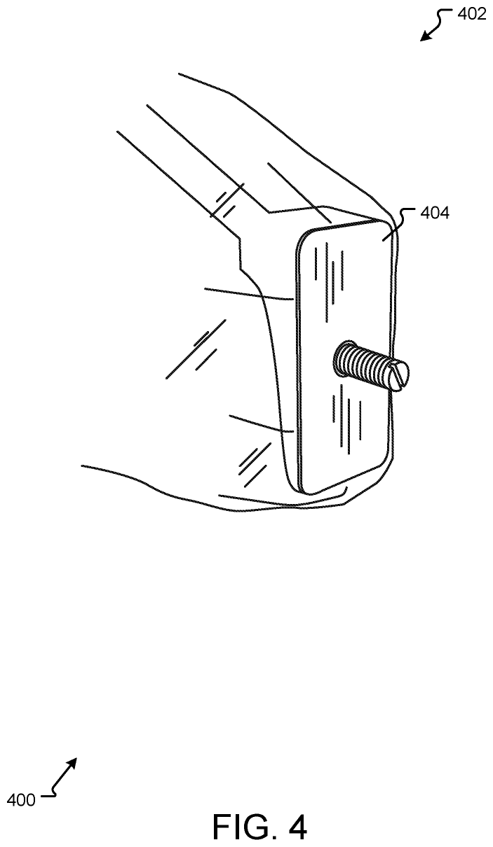

FIG. 4 shows a diagram 400 of an interface block 402 with a liner plate 404 attached according to at least one embodiment of the present disclosure. In some examples, the interface block 402 may be an example of the interface block 302 as described with reference to FIG. 3. As shown in FIG. 4, the liner plate 404 may fit over a mount surface of the interface block 402. In some examples, the liner plate may be added to or may attach to a sterile drape covering the interface block 402 (e.g., via glue or other types of adhesives). The liner plate 404 may assist in preventing tears from occurring in a sterile drape when an end-effector block is attached to the interface block 402. As described previously with reference to FIG. 3, the interface block 402 may include a set of kinematic attachment features, where the end-effector block includes a set of kinematic connectors that engage or work with the set of kinematic attachment features of the interface block 402. However, the set of kinematic connectors may cause tears or holes in the sterile drape as the end-effector block is moved, rotated, actuated, etc., which could compromise sterilization.

Accordingly, the liner plate 404 may reduce the chance that these tears or holes are formed by distributing forces exerted by the end-effector block on the mounting surface of the interface block 402. In some examples, the liner plate 404 may include kinematics “connection divots” that match and account for the set of kinematic features of the interface block 402 or may be flat but can be deformed without tearing or tearing the sterile drape. In some examples, the liner plate 404 may include a clearance hole that is arranged such that a threaded rod of the interface block 402 passes through the clearance hole. Additionally, the liner plate 404 may be attached to the interface block 402 such that an area of the sterile drape is disposed between the liner plate 404 and the mount surface of the interface block 402. In some examples, the liner plate 404 may be made from a flat metal plate material corresponding to at least one of aluminum, copper, titanium, cobalt-chrome, and stainless steel (e.g., or a different metal not explicitly listed herein). Additionally or alternatively, the liner plate 404 may be made from a flat polymer plate material (e.g., plastic material).

FIG. 5A shows a perspective view 500 of a surgical system 502 according to at least one embodiment of the present disclosure. The surgical system 502 may include an interface block disposed at a distal end of a robot arm. As shown in FIG. 5A, an end-effector block 504 may attach to the interface block. As described previously, the end-effector block 504 may include a set of kinematic connectors that are arranged (e.g., on the rear of the end-effector block 504 that is not shown) to engage with a set of kinematic attachment features of the interface block.

Additionally, the end-effector block 504 may include a mount hole 506 passing through the end-effector block 504. In some examples, the mount hole 506 may include an unthreaded clearance hole and counterbore. In some examples, the end-effector block 504 is moveable between an attached state and a detached state with respect to the interface block. For example, in the attached state, the set of kinematic connectors of the end-effector block 504 are engaged with the set of kinematic attachment features of the interface block, a threaded rod of the interface block is disposed in the mount hole 506, and a nut threadedly engages with the threaded rod clamping the end-effector block 504 against the interface block. Additionally or alternatively, in the detached state, the nut is removed and the end-effector block 504 is separated from the interface block such that the threaded rod remains attached to the interface block and is no longer disposed in the mount hole 506. That is, the end-effector block 504 may be moveable between the attached state and the detached state without removing the threaded rod from the interface block and without exposing an environment inside the sterile drape to an environment outside of the sterile drape.

The end-effector block 504 may also include a body 508 and a tool receiving aperture 510. The tool receiving aperture 510 may be designed to accommodate or handle any number of surgical tools for use in robotic surgical systems.

FIG. 5B shows a front view 501 of the surgical system 502 according to at least one embodiment of the present disclosure. As shown in FIG. 5B and described previously, a nut 512 is used to threadedly engage with the threaded rod of the interface block to clamp the end-effector block 504 against the interface block. In some examples, the nut 512 can be threaded onto the threaded rod and/or removed from the threaded rod by using the access granted by the mount hole 512. For example, a nut driver (e.g., or a screwdriver with a hex socket) may fit through the mount hole 512 to be able to thread or unthread the nut 512 from the threaded rod. In some examples, the nut 512 may have a socket for or used by a specific tool (e.g., key, screwdriver, unique tool, etc.) that prevents opening the threaded rod (e.g., screw) while opening the nut 512.

By using the nut 512 to secure the end-effector block 504 to the interface block, the end-effector block 504 can be easily replaced mid-operation with another end-effector block or a different tool for the operation. For example, the surgical system 502 and method of interconnecting the end-effector block 504 described herein allows an arm guide/end-effector to be connected/disconnected using a nut-to-rod interface rather than an external screw-in-hole interface. This reversed interface allows changeover of an arm guide/end-effector without compromising a sterile barrier between the patient and the surgical system 502.

FIG. 6 shows an assembly diagram 600 for attaching an end-effector block 604 to an interface block 602 according to at least one embodiment of the present disclosure. For example, as described previously with reference to FIGS. 5A and 5B, a nut 606 threadedly engages with a threaded rod of the interface block 602 clamping the end-effector block 604 against the interface block 602, where the nut 606 threads onto the threaded rod through a mount hole of the end-effector block 604.

While the end-effector block 604 is shown and described herein as being attached to the interface block 602 using the nut 606 to engage with the threaded rod of the interface block 602, other mechanisms and techniques may be used to attach the end-effector block 604 to the interface block 602 not explicitly described or illustrated herein. For example, the end-effector block 604 may attach to the interface block 602 using one or more magnets or electro-magnets, a latch, an eccenter/eccentric handle, etc.

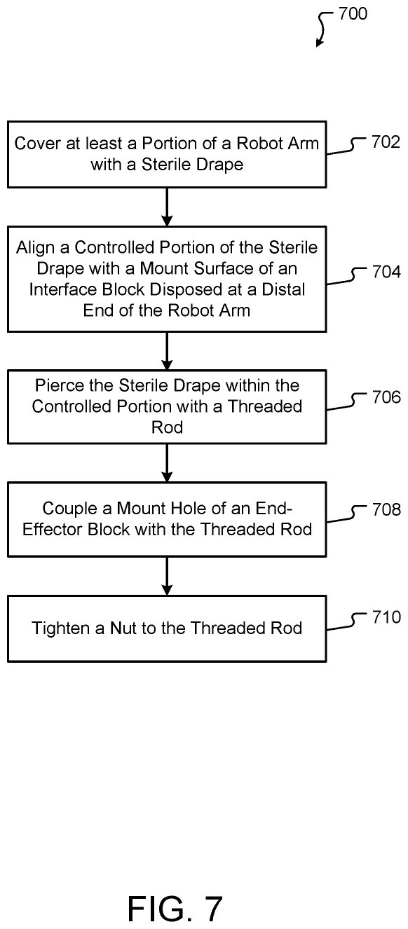

FIG. 7 depicts a method 700 that may be used, for example, to selectively couple an end-effector block to a robot arm in a sterile environment.

The method 700 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 700. The at least one processor may perform the method 700 by executing elements stored in a memory such as the memory 106. The elements stored in the memory and executed by the processor may cause the processor to execute one or more steps of a function as shown in method 700. One or more portions of a method 700 may be performed by the processor executing any of the contents of memory, such as an image processing 120, a segmentation 122, a transformation 124, and/or a registration 128.

The method 700 comprises covering at least a portion of the robot arm with a sterile drape (step 702). In some examples, the sterile drape is intended to provide a sterile barrier between the robot arm and the patient (e.g., and the environment surrounding the robot arm).

The method 700 also comprises aligning a controlled portion of the sterile drape with a mount surface of an interface block disposed at a distal end of the robot arm (step 704). In some examples, aligning the controlled portion of the sterile drape may include aligning a gasket surrounding the controlled portion of the sterile drape with a hole disposed in the mount surface and adhering the gasket to the mount surface such that the mount hole is surrounded by the gasket and an axis of the mount hole is disposed within the controlled portion of the sterile drape.

The method 700 also comprises piercing the sterile drape within the controlled portion with a threaded rod such that the threaded rod is fastened to the interface block and extends a distance from the mount surface through the sterile drape (step 706). In some examples, a liner plate may be attached to the mount surface of the interface block. For example, the liner plate may include a clearance hole for attaching the liner plate to a portion of the sterile drape such that the threaded rod passes through the clearance hole and an area of the sterile drape is sandwiched between the liner plate and the mount surface of the interface block.

The method 700 also comprises coupling a mount hole of the end-effector block with the threaded rod such that the threaded rod is disposed within the mount hole (step 708). For example, the end-effector block is placed up against the interface block, such that the mount hole of the end-effector block is aligned with the threaded rod of the interface block.

The method 700 also comprises tightening a nut to the threaded rod such that the nut clamps the end-effector block in a fastened state against the interface block (step 710). In some examples, after the nut is tightened, tools may be placed into a tool receiving aperture of the end-effector block to perform different operations (e.g., as part of a robotic surgical operation). If a different end-effector block and/or tool is needed for a subsequent operation, the nut may be loosened apart from the threaded rod, and the end-effector block may be removed from the threaded rod and interface block while the threaded rod remains attached to the interface block and while the sterile drape remains in place. Subsequently, a mount hole of a second end-effector block may be coupled with the threaded rod such that the threaded rod is disposed within the mount hole of the second end-effector block, and the nut may be tightened again to the threaded rod such that the nut clamps the second end-effector block in a fastened state against the interface block.

The present disclosure encompasses embodiments of the method 700 that comprise more or fewer steps than those described above, and/or one or more steps that are different than the steps described above.

As noted above, the present disclosure encompasses methods with fewer than all of the steps identified in FIG. 7 (and the corresponding description of the method 700), as well as methods that include additional steps beyond those identified in FIG. 7 (and the corresponding description of the method 700). The present disclosure also encompasses methods that comprise one or more steps from one method described herein, and one or more steps from another method described herein. Any correlation described herein may be or comprise a registration or any other correlation.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A surgical system, comprising:
a robot arm comprising a proximal end and a distal end;
an interface block disposed at the distal end, the interface block comprising:
a mount surface;
a threaded rod extending a distance from the mount surface; and
a set of kinematic attachment features disposed on the mount surface around the threaded rod; and
an end-effector block, comprising:
a set of kinematic connectors that are arranged to engage with the set of kinematic attachment features of the interface block; and
a mount hole passing through the end-effector block, wherein the end-effector block is moveable between an attached state and a detached state, wherein, in the attached state, the set of kinematic connectors are engaged with the set of kinematic attachment features, the threaded rod is disposed in the mount hole, and a nut threadedly engages with the threaded rod clamping the end-effector block against the interface block.

2. The surgical system of claim 1, wherein, in the detached state, the nut is removed and the end-effector block is separated from the interface block such that the threaded rod remains attached to the interface block and is no longer disposed in the mount hole.

3. The surgical system of claim 2, further comprising:
a sterile drape that covers the robot arm, wherein the threaded rod pierces a controlled portion of the sterile drape; and
a liner plate comprising a clearance hole that is arranged such that the threaded rod passes through the clearance hole and an area of the sterile drape is disposed between the liner plate and the mount surface, and wherein the end-effector block is moveable between the attached state and the detached state without removing the threaded rod from the interface block and without exposing an environment inside the sterile drape to an environment outside of the sterile drape.

4. The surgical system of claim 3, wherein the controlled portion of the sterile drape is disposed within a periphery of the interface block.

5. The surgical system of claim 4, wherein the sterile drape further comprises:
a gasket attached to the sterile drape, the gasket surrounding the controlled portion of the sterile drape and an outer circumference of the threaded rod.

6. The surgical system of claim 5, wherein the gasket is formed in a flat circular ring shape, and wherein at least one flat surface of the gasket comprises an adhesive layer.

7. The surgical system of claim 6, wherein the gasket is attached to the sterile drape on a side of the sterile drape facing the mount surface, and wherein the adhesive layer is disposed in contact with the mount surface sealing the environment inside the sterile drape and around the gasket from the environment outside of the sterile drape.

8. The surgical system of claim 6, wherein the liner plate is made from a flat metal plate material corresponding to at least one of aluminum, copper, titanium, cobalt-chrome, and stainless steel.

9. The surgical system of claim 6, wherein the liner plate is made from a flat polymer plate material.

10. The surgical system of claim 6, wherein the mount hole comprises an unthreaded clearance hole and counterbore.

11. A robot end-effector mount system, comprising:
an interface block, comprising:
a mount surface;
a threaded rod protruding from the mount surface; and
an end-effector block, comprising:
a body;
a tool receiving aperture; and
a mount hole passing through the body, wherein the end-effector block is moveable between an attached state and a detached state with the interface block, wherein, in the attached state, the threaded rod is disposed in the mount hole and a nut threadedly engages with the threaded rod clamping the end-effector block against the interface block, and wherein the end-effector block is moveable between the attached state and the detached state without removing the threaded rod from the interface block.

12. The robot end-effector mount system of claim 11, wherein, in the detached state, the nut is removed and the end-effector block is separated from the interface block such that the threaded rod remains attached to the interface block and is no longer disposed in the mount hole.

13. The robot end-effector mount system of claim 12, further comprising:
a sterile drape that covers the interface block, wherein the sterile drape is pierced at a controlled portion by the threaded rod; and
a liner plate comprising a clearance hole that is arranged such that the threaded rod passes through the clearance hole and an area of the sterile drape is disposed between the liner plate and the mount surface, and wherein the end-effector block is moveable between the attached state and the detached state without exposing an environment inside the sterile drape on a side of the sterile drape facing the mount surface to an environment outside of the sterile drape on a side of the sterile drape facing the end-effector block.

14. The robot end-effector mount system of claim 13, wherein the controlled portion of the sterile drape is disposed within an outer edge of the interface block.

15. The robot end-effector mount system of claim 14, wherein the sterile drape further comprises:
a gasket attached to the sterile drape, wherein the gasket surrounds the controlled portion of the sterile drape and an outer circumference of the threaded rod, wherein the gasket is formed in a flat circular ring shape, and wherein at least one flat surface of the gasket comprises an adhesive layer.

16. The robot end-effector mount system of claim 15, wherein the gasket is formed in a flat circular ring shape, and wherein at least one flat surface of the gasket comprises an adhesive layer.

17. The robot end-effector mount system of claim 16, wherein the gasket is attached to the sterile drape on a side of the sterile drape facing the mount surface, and wherein the adhesive layer is disposed in contact with the mount surface sealing the environment inside the sterile drape and around the gasket from the environment outside of the sterile drape.

18. The robot end-effector mount system of claim 16, wherein the liner plate is made from a flat metal plate material corresponding to at least one of aluminum, copper, titanium, cobalt-chrome, and stainless steel.

19. The robot end-effector mount system of claim 16, wherein the liner plate is made from a flat polymer plate material.

20. A robot end-effector mount system, comprising:
an end-effector block, comprising:
a body;
a tool receiving aperture; and
a mount hole passing through the body, wherein the end-effector block is moveable between an attached state and a detached state with an interface block, wherein, in the attached state, a threaded rod is disposed in the mount hole and a nut threadedly engages with the threaded rod clamping the end-effector block against the interface block, and wherein the end-effector block is moveable between the attached state and the detached state without removing the threaded rod from the interface block.
